(19) United States
(12) Patent Application Publication
(10) Pub. No.: US 2014/0194477 A1
(43) Pub. Date: Jul. 10, 2014

(54) KINASE PROTEIN BINDING INHIBITORS

(71) Applicant: Roswell Park Cancer Institute, Buffalo, NY (US)

(72) Inventors: William G. Cance, Orchard Park, NY (US); Vita Golubovskaya, Orchard Park, NY (US); Elena V. Kurenova, West Falls, NY (US)

(73) Assignee: Roswell Park Cancer Institute, Buffalo, NY (US)

(21) Appl. No.: 14/048,963

(22) Filed: Oct. 8, 2013

Related U.S. Application Data
(63) Continuation of application No. PCT/US2012/032524, filed on Apr. 6, 2012.
(60) Provisional application No. 61/473,642, filed on Apr. 8, 2011.

(51) Int. Cl. A61K 31/4402; A61K 31/4188; A61K 31/136

(52) U.S. Cl. CPC A61K 31/4402; A61K 31/136; A61K 31/4188
USPC 514/352; 514/646; 514/393

(57) ABSTRACT

The invention relates to phosphorylation inhibitor compounds and methods of identifying and using them. The invention further relates to pharmaceutical compositions and methods for treating cell proliferative disorders, especially cancer.

### CROSS REFERENCE TO RELATED APPLICATIONS

This application claims the benefit of PCT/US2012/032524, filed Apr. 6, 2012, which claims priority benefit of U.S. Provisional Patent Application No. 61/473,642, filed Apr. 8, 2011, the contents of which is hereby incorporated by reference in its entirety.

### STATEMENT OF RIGHTS TO INVENTIONS MADE UNDER FEDERALLY SPONSORED RESEARCH

This work was supported in part by a National Institutes of Health/NCI Grant, Grant No. 2-ROI-CA65910-09-13. The government has certain rights in the invention.

### BACKGROUND OF THE INVENTION

Focal Adhesion Kinase (FAK) is an important survival molecule that is upregulated in a broad range of solid tumors and is expressed at very low levels in normal tissues, creating a therapeutic window and making this protein a highly attractive target for the treatment of cancer See, e.g., WO 2005/049852, the contents of which are incorporated by reference. The market for novel drug therapy targeting cancers of the breast, colon, pancreas, and thyroid is extensive. According to the American Cancer Society, it is estimated that 425,000 new cases of these cancers will be diagnosed this year in this country alone. Cancer drug therapy is an existing major product line of several pharmaceutical companies, and the development of drugs targeting FAK would be a natural complement to their existing products.

FAK is overexpressed in many cancer types compared to other kinase targets. Compounds that target FAK could be prescribed for many cancer types including breast, colon, pancreas, thyroid, lung, and melanoma.

Several groups are exploring the targeting of FAK as potential cancer therapeutics. The targeting of FAK typically has been focused on the kinase domain of FAK. This approach has proven unsuccessful as disruption of the kinase domain does not specifically interfere with the signaling downstream of FAK and other related tyrosine kinases have been affected by the drugs. Delineated herein is a novel approach that focuses on FAK phosphorylation.

FAK is a 125 kDa protein that localizes to focal adhesions (1) and is activated and tyrosine phosphorylated in response to integrin clustering (2). Tyrosine 397 is an autophosphoiylation site of FAK and is a critical component in downstream signaling (3), providing a high-affinity binding site for the SH2 domain of Src family kinases (4), (5). The interaction between Y397-activated FAK and Src leads to a cascade of tyrosine phosphorylation of multiple sites in FAK (-576, -577, -925), as well as other signaling molecules such as p130^{CAS} and paxillin, resulting in cytoskeletal changes and activation of other downstream signaling pathways (6). Y397 is also a site of binding P13 kinase, growth factor receptor binding Grb-7, She, and other proteins. Thus, the Y397 site is one of the main phosphorylation sites that activate FAK signaling in the cells.

Focal adhesion kinase is involved in multiple cellular functions such as cell proliferation, survival, motility, invasion, metastasis, and angiogenesis (7). Different approaches to inhibit FAK with FAK anti-sense oligonucleotides (8), dominant-negative C-terminal domain of FAK, FAK-CD or FRNK (9,10) or FAK siRNA (11), (12) caused decreased cellular viability, growth inhibition or apoptosis. Recently, FAK has been proposed to be a new potential therapeutic target in cancer (13,14). Two novel kinase inhibitors of FAK, blocking FAK catalytic activity, were developed and reported recently, one by Novartis: NVP-TAE226 (15) (16) and another by Pfizer: PF-573,228 (17). The first inhibitor, TAE226, inhibited glioma and ovarian tumor growth in vivo (16,18), although it also inhibited IGFR kinase (16). The efficacy of the PF-573,228 on tumor growth in vivo has not been reported, it inhibited only motility and did not inhibit cell growth and survival in vitro (17), Both of these inhibitors effectively blocked Y397-FAK phosphorylation.

We found that certain compounds, including 1,2,4,5-benzenetetraamine tetrahydrochloride, called Y15, targets the Y397 site, directly and specifically decreases Y397-phosphorylation of FAK in vitro, inhibits cancer cell viability in vitro, causes detachment, decreases cell adhesion and blocks tumor growth in vivo. We also found that certain compounds, including N′-[(4-chlorophenyl)methylj-N,N-dimethyl-N′-pyridin-2-yl-ethane-1,2-diamine (NSC 409949; Sigma C1915, Suprastin; chloropyramine hydrochloride), called C4, modulates (e.g., inhibiting or stimulating) (directly or indirectly) FAK binding activity, and C4 in various assays indicates that it specifically dephosphorylates FAK.

In particular, it has been determined that compounds herein (e.g., C4, Y15) exhibit synergistic efficacy in combination with temozolomide.

### SUMMARY OF THE INVENTION

In one aspect, the invention provides a method of treating a subject suffering from or susceptible to a brain cancer comprising administering to the subject in need thereof a therapeutically effective amount of the compound C4.

In one aspect, the invention provides a method of treating a subject suffering from or susceptible to a brain cancer comprising administering to the subject in need thereof a therapeutically effective amount of the compound Y15.

In one aspect, the invention provides a method of treating a subject suffering from or susceptible to melanoma comprising administering to the subject in need thereof a therapeutically effective amount of the compound C4.

In one aspect, the invention provides a method of treating a subject suffering from or susceptible to melanoma comprising administering to the subject in need thereof a therapeutically effective amount of the compound Y15.

In aspects, the methods herein further comprise administering to the subject in need thereof a therapeutically effective amount of temozolomide.

In aspects, the methods include those further comprising wherein the disease, disorder or symptom thereof (i.e., brain cancer, melanoma, glioblastoma multiforme) is ameliorated and/or treated.

In aspects, the compounds are administered separately.

In aspects, the compounds are administered concurrently.

In aspects, the compounds are administered in a single-dose form.

Another aspect is a composition comprising temozolomide and a second compound wherein the second compound is:

C4: N′-[(4-chlorophenyl)methyl]-N,N-dimethyl-N-pyridin-2-yl-ethane-1,2-diamine (NSC 409949; Sigma C1915, Suprastin; chloropyramine hydrochloride); or

Y15: 1,2,4,5-benzenetetraamine tetrahydrochloride.

In aspects, the composition further comprises a pharmaceutically acceptable carrier.

In aspects, the composition is in a single-dose formulation.

Another aspect is a kit comprising temozolomide and a second compound that is C4 or Y15 and instructions for administration of the compounds to a subject identified as in need of treatment for a disease, disorder, or symptom thereof. In aspects, the kit includes instructions wherein the disease, disorder, or symptom thereof is brain cancer, melanoma, or glioblastoma multiforme.

In one aspect, the invention provides a method of treating a subject suffering from or susceptible to a cell proliferative disorder comprising administering to subject in need thereof a therapeutically effective amount of a compound capable of modulating FAK protein-protein binding interactions. In one embodiment, the compound is capable of binding to or interacting with a binding pocket that affects FAK binding. In another embodiment, the compound is capable of binding to or interacting with a binding pocket that affects FAK phosphorylation.

### BRIEF DESCRIPTION OF THE DRAWINGS

The present invention is further described below with reference to the following non-limiting examples and with reference to the following figures, in which:

FIG. 1. illustrates treatment of DBTRG cells with C4

FIG. 2. illustrates treatment of U-87 MG cells with C4

FIG. 3. illustrates treatment of U-87 MG cells with C4 and temozolomide

FIGS. 4-7 illustrate treatment of U-87 MG cells with Y15 and temozolomide

FIGS. 8-9 illustrate treatment of DBTRG cells with Y15 and temozolomide

FIGS. 10-11 illustrate treatment of SC61 cells with Y15 and temozolomide

FIGS. 12-13 illustrate treatment of U-251 cells with Y15 and temozolomide

FIG. 14. illustrates C4 inhibition of tumor growth in a glioblastoma mouse model as a single agent—tumor volume

FIG. 15. illustrates C4 inhibition of tumor growth in a glioblastoma mouse model as a single agent—tumor weight

FIG. 16. illustrates C4 inhibition of tumor growth in a glioma singenic mouse model as a single agent—tumor weight

NOTE: in FIGS.*=statistically significant

### DETAILED DESCRIPTION OF THE INVENTION

In particular, it has been determined that compounds herein (e.g., C4, Y15) exhibit efficacy in treating brain cancer, melanoma, and glioblastoma multiforme, and in particular synergistic efficacy in combination with temozolomide for treating brain cancer, melanoma, and glioblastoma multiforme. Thus the invention relates to compositions and methods of using them to treat brain cancer, melanoma, and glioblastoma multiforme in subjects.

### 1. Definitions

Before further description of the present invention, and in order that the invention may be more readily understood, certain terms are first defined and collected here for convenience.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C1-C30 for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, atylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylearbonylamino, arylcarbonylamino, carbamoyl and ureido), amidino, imino, sulfhydryl, alkylthio, arylthio, thiocarboxylate, sulfates, sulfonato, sulfamoyl, sulfonamide, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenyhnethyl(benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C1-C4 alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like, Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “associating with” refers to a condition of proximity between a chemical entity or compound, or portions thereof, and a binding pocket or binding site on a protein. The association may be non-covalent (wherein the juxtaposition is energetically favored by hydrogen bonding or van der Waals or electrostatic interactions) or it may be covalent.

The term “binding pocket”, as used herein, refers to a region of a molecule or molecular complex, that, as a result of its shape, favorably associates with another chemical entity or compound.

The language “biological activities” of a compound of the invention includes all activities elicited by compound of the inventions in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their mirror image partner.

The term “diastereomers” refers to stereoisomers with two or inure centers of dissymmetry and whose molecules are not mirror images of one another,

The term “effective amount” includes an amount effective, at dosages and for periods of time necessary, to achieve the desired result, e.g., sufficient to treat a cell proliferative disorder. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.” The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the present invention.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced toxicity.

The term “cell proliferative disorder” includes disorders involving the undesired or uncontrolled proliferation of a cell. Examples of such disorders include, but are not limited to, tumors or cancers (e.g., lung (small cell and non-small cell), thyroid, prostate, pancreatic, breast or colon), sarcoma or melanoma.

The language “a FAK protein-protein binding partner” refers to a protein (including those delineated herein) that bind with FAK (e.g., full length, N-terminus, C-terminus, carboxy terminus, kinase domain, FERM domain, FAT domain).

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈ alkenyl, C₂-C₈alkoxy, C₂-C₈alkyl ether, C₃-C₈alkanone, C₁-C₈alkylthio, amino, mono- or di-(C1-C₈alkyl)amino, haloC₁-C₈alkyl, haloC₁-C₈alkoxy, C₁-C₈alkanoyl, C₂-C₈alkanoyloxy, C₁-C_{C}₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “isomers” or “stereoisomers” refers to compounds which have identical chemical constitution, but differ with regard to the arrangement of the atoms or groups in space.

The term “modulate” refers to an increase or decrease, e.g., in the ability of a cell to proliferate in response to exposure to a compound of the invention, e.g., the inhibition of proliferation of at least a sub-population of cells in an animal such that a desired end result is achieved, e.g., a therapeutic result.

The term “obtaining” as in “obtaining a compound capable of inhibiting Y397 phosphorylation of FAK” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, acylamino, diarylamino, and alkylatylamino), acylamino (including alkylcarbonylamino, arylcarbonylamino, carbamoyl and ureido), amidino, imino, sulthydryl, alkylthio, arylthio, thiocarboxylate, sulfates, sulfonato, sulfamoyl, sulfonamido, nitro, trifluoromethyl, cyano, azido, heterocyclyl, alkyl, alkylaiyl, or an aromatic or heteroaromatic moiety. /

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention any formula herein or otherwise described herein which is effective, upon single or multiple dose administration to the patient, in preventing or treating a cell proliferative disorder.

The language “reduced toxicity” is intended to include a reduction in any undesired side effect elicited by a compound of the invention when administered in vivo.

The term “sulthydryl” or “thiol” means —SH.

The term “subject” includes organisms which are capable of suffering from a cell proliferative disorder or who could otherwise benefit from the administration of a compound of the invention of the invention, such as human and non-human animals. Preferred humans include human patients suffering from or prone to suffering from a cell proliferative disorder or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The term “susceptible to a cell proliferative disorder” is meant to include subjects at risk of developing disorder of cell proliferation, e.g., cancer, i.e., subjects suffering from viral infection with cancer viruses, subjects that have been exposed to ionizing radiation or carcinogenic compounds, subjects having a family or medical history of cancer, and the like. /

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of a compound of the invention of the invention refers to an amount of an agent which is effective, upon single or multiple dose administration to the patient, in inhibiting cell proliferation and/or symptoms of a cell proliferative disorder, or in prolonging the survivability of the patient with such a cell proliferative disorder beyond that expected in the absence of such treatment.

With respect to the nomenclature of a chiral center, terms “d” and “I” configuration are as defined by the IUPAC Recommendations. As to the use of the terms, diastereomer, racemate, epimer and enantiomer will be used in their normal context to describe the stereochemistry of preparations.

### 2. Compounds of the Invention

In one aspect, the invention provides compounds capable of modulating (e.g., inhibiting or stimulating) (directly or indirectly) FAK binding activity.

In one embodiment, the invention provides a compound capable of modulating FAK protein-protein binding; and pharmaceutically acceptable esters, salts, and prodrugs thereof.

Certain preferred compounds include compounds specifically delineated herein:

C 1: 2-[2-(anilinocarbamoyl)phenyl]benzoic acid;

C2: N′-[(4-chlorophenyl)methyl]-N,N-dimethyl-N′-pyridin-2-yl-ethane-1,2-diamine;

C3: pyridin-2-ylmethanamine;

C4: N′-[(4-chlorophenyl)methyl]-N,N-dimethyl-N′-pyridin-2-yl-ethane-1,2-diamine (NSC 409949; Sigma C 1915, Suprastin; chloropyramine hydrochloride);

C5: 1-(3-fluorophenyl)-3-naphthalen-2-yl-urea (NSC 216201);

C6: N44-[(3-fluorophenyl)carbamoylamino]phenyl]lacetamide;

C7: N44-[(4-fluorophenyl)carbamoylamino]phenyl]acetamide;

C8: N-[(6-nitrobenzo[1,3]dioxol-5-yOmethylideneamino]benzamide;

C9: N-[1-(4-chlorophenyl)propyd-N-ethyl-pyridin-2-amine;

C 10: 10-(4-chlorophenyl)-3-methyl-7-(5-methylpyridin-2-yl)-8-oxa-1,7,9-triazabicyclo[4.4.0]deca-2,4,9-triene;

C 11: 2-(1,7-diazabicyclo[4.3.0]nona-2,4,6,8-tetraen-8-yDacetic acid;

C12: 2-(4-methyl-1,7-diazabicyclo[4.3.0]nona-2,4,6,8-tetraen-8-yl)acetic acid;

C27: usinic acid derivative 4, 4a-dihydro-4A(phenylthio), racemate (NSC250435);

Y11: 3,5,7-triaza-1-azonlatricyclo(3.3.1.13,7)decane, 1-(2-hycloxyethyl)-, bromide;

Y15: 1,2,4,5-benzenetetraamine tetrahydrochloride;

Y30: 9-thin-1,3,6,8-tetraazatricyclo[4.3.1.1(3,8)]undecane, 9,9-dioxide;

The invention also relates to the pharmaceutically acceptable salts and esters of the above-mentioned compounds.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomerie salts.

3. Uses of the compounds of the Invention

In one embodiment, the invention provides methods for treating a subject for a cell proliferative disorder, by administering to the subject an effective amount of a compound delineated herein. A cell proliferative disorder includes cancer. In certain embodiments, the subject is a mammal, e.g., a primate, e.g., a human.

In certain embodiments, the methods of the invention include administering to a subject a therapeutically effective amount of a compound of the invention in combination with another pharmaceutically active compound. Examples of pharmaceutically active compounds include compounds known to treat cell proliferative disorders, e.g., anticancer agent, antiproliferative agent, chemotherapeutic. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J, Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

In certain embodiments, the compound of the invention can be used in combination therapy with conventional cancer chemotherapeutics. Conventional treatment regimens for leukemia and for other tumors include radiation, drugs, or a combination of both. In addition to radiation, the following drugs, usually in combinations with each other, are often used to treat acute leukemias: vincristine, prednisone, methotrexate, mercaptopurine, cyclophosphamide, and cytarabine. Other examples include, for example, doxorubicin, cisplatin, taxol, 5-fluorouracil, etoposid, gemcitabine, etc., which demonstrate advantages (e.g., chemosensitization of cells) in combination with the compounds described herein. In chronic leukemia, for example, busulfan, melphalan, and chlorambucil can be used in combination. Most conventional anti-cancer drugs are highly toxic and tend to make patients quite ill while undergoing treatment. Vigorous therapy is based on the premise that unless every cancerous cell is destroyed, the residual cells will multiply and cause a relapse.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method). In other methods, the subject is prescreened or identified as in need of such treatment by assessment for a relevant marker or indicator of suitability for such treatment.

Methods delineated herein include those wherein the subject is identified as in need of a particular stated treatment and wherein upon administration of a compound or composition delineated herein is treated. In another aspect, the disease, disorder or symptom thereof identified in the subject is ameliorated, reduced or deemed in remission after administration of a compound or composition delineated herein.

Determination of a therapeutically effective anti-proliferative amount or a prophylactically effective anti-proliferative amount of the compound of the invention of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective anti-proliferative amount or dose, and the prophylactically effective anti-proliferative amount or dose, a number of factors are considered by the attending clinician, including, but not limited to: the specific cell proliferative disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective anti-proliferative amount of a compound of the invention of the invention is expected to vary from about 0.1 milligram per kilogram of body weight per day (mg/kg/day) to about 100 mg/kg/day.

Compounds determined to be effective for the prevention or treatment of cell proliferative disorders in animals, e.g., dogs, chickens, and rodents, may also be useful in treatment of tumors in humans. Those skilled in the art of treating tumors in humans will know, based upon the data obtained in animal studies, the dosage and route of administration of the compound to humans. In general, the dosage and route of administration in humans is expected to be similar to that in animals.

The identification of those patients who are in need of prophylactic treatment for cell proliferative disorders is well within the ability and knowledge of one skilled in the art. Certain of the methods for identification of patients which are at risk of developing cell proliferative disorders which can be treated by the subject method are appreciated in the medical arts, such as family history, and the presence of risk factors associated with the development of that disease state in the subject patient. A clinician skilled in the art can readily identify such candidate patients, by the use of; for example, clinical tests, physical examination and medical/family history. A method of assessing the efficacy of a treatment in a subject includes determining the pre-treatment extent of a cell proliferative disorder by methods well known in the art (e.g., determining tumor size or screening for tumor markers where the cell proliferative disorder is cancer) and then administering a therapeutically effective amount of an inhibitor of cell proliferation (e.g., those described herein) according to the invention to the subject. After an appropriate period of time after the administration of the compound (e.g., 1 day, 1 week, 2 weeks, one month, six months), the extent of the cell proliferative disorder is determined again. The modulation (e.g., decrease) of the extent or invasiveness of the cell proliferative disorder indicates efficacy of the treatment. The extent or invasiveness of the cell proliferative disorder may be determined periodically throughout treatment. For example, the extent or invasiveness of the cell proliferative disorder may be checked every few hours, days or weeks to assess the further efficacy of the treatment. A decrease in extent or invasiveness of the cell proliferative disorder indicates that the treatment is efficacious. The method described may be used to screen or select patients that may benefit from treatment with an inhibitor of a cell proliferative disorder.

As used herein, “obtaining a biological sample from a subject,” includes obtaining a sample for use in the methods described herein. A biological sample is described above.

In another aspect, a compound of the invention is packaged in a therapeutically effective amount with a pharmaceutically acceptable carrier or diluent. The composition may be formulated for treating a subject suffering from or susceptible to a cell proliferative disorder, and packaged with instructions to treat a subject suffering from or susceptible to a cell proliferative disorder.

In another aspect, the invention provides methods for inhibiting cell proliferation. In one embodiment, a method of inhibiting cell proliferation (or a cell proliferative disorder) according to the invention includes contacting cells with a compound capable of modulating FAK, FAK binding partner, or specific domains thereof. In either embodiment, the contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

In another aspect, methods of inhibiting a cell proliferative disorder in a subject include administering an effective amount of a compound of the invention (i.e., a compound described herein) to the subject. The administration may be by any route of administering known in the pharmaceutical arts. The subject may have a cell proliferative disorder, may be at risk of developing a cell proliferative disorder, or may need prophylactic treatment prior to anticipated or unanticipated exposure to a conditions capable of increasing susceptibility to a cell proliferative disorder, e.g., exposure to carcinogens or to ionizing radiation.

In one aspect, a method of monitoring the progress of a subject being treated with a compound herein includes determining the pre-treatment status (e.g., size, growth rate, or invasiveness of a tumor) of the cell proliferative disorder, administering a therapeutically effective amount of a compound herein to the subject, and determining the status (e.g., size, growth rate, or invasiveness of a tumor) of the cell proliferative disorder after an initial period of treatment with the compound, wherein the modulation of the status indicates efficacy of the treatment.

The subject may be at risk of a cell proliferative disorder, may be exhibiting symptoms of a cell proliferative disorder, may be susceptible to a cell proliferative disorder and/or may have been diagnosed with a cell proliferative disorder.

If the modulation of the status indicates that the subject may have a favorable clinical response to the treatment, the subject may be treated with the compound. For example, the subject can be administered therapeutically effective dose or doses of the compound.

Kits of the invention include kits for treating a cell proliferative disorder in a subject. The kit may include a compound of the invention, for example, a compound described herein, pharmaceutically acceptable esters, salts, and prodrugs thereof, and instructions for use. The instructions for use may include information on dosage, method of delivery, storage of the kit, etc. The kits may also include, reagents, for example, test compounds, buffers, media (e.g., cell growth media), cells, etc. Test compounds may include known compounds or newly discovered compounds, for example, combinatorial libraries of compounds. One or more of the kit of the invention may be packaged together, for example, a kit for assessing the efficacy of an treatment for a cell proliferative disorder may be packaged with a kit for monitoring the progress of a subject being treated for a cell proliferative disorder according to the invention.

The present methods can be performed on cells in culture, e.g. in vitro or ex vivo, or on cells present in an animal subject, e.g., in vivo. Compounds of the inventions can be initially tested in vitro using primary cultures of proliferating cells, e.g., transformed cells, tumor cell lines, and the like.

The present method can be performed on cells in culture, e.g. in vitro or ex vivo, or on cells present in an animal subject, e.g., in vivo. Compound of the invention can be initially tested in vitro using cells from the respiratory tract from embryonic rodent pups (See e.g. U.S. Pat. No. 5,179,109—fetal rat tissue culture), or other mammalian (See e.g. U.S. Pat. No. 5,089,517—fetal mouse tissue culture) or non-mammalian animal models.

Alternatively, the effects of compound of the invention can be characterized in vivo using animals models.

### 4. Pharmaceutical Compositions

The invention also provides a pharmaceutical composition, comprising an effective amount of a compound described herein and a pharmaceutically acceptable carrier. In a further embodiment, the effective amount is effective to treat a cell proliferative disorder, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (I) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (₃) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, more preferably from about 10 per cent to about 30 per cent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (I) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (₃) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form, Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the compound of the invention for the present invention is the maximum that a patient can tolerate and not develop serious side effects. Preferably, the compound of the invention of the present invention is administered at a concentration of about 0.001 mg to about 100 mg per kilogram of body weight, about 0.001—about 10 mg/kg or about 0.001 mg—about 100 mg/kg of body weight. Ranges intermediate to the above-recited values are also intended to be part of the invention.

### EXAMPLES

The invention is further illustrated by the following examples which are intended to illustrate but not limit the scope of the invention.

### Materials

Cell lines—U87 MG and DBTRG-O5MG (Denver Brain Tumor Research Group 05) brain cancer cell lines are available from ATCC. References relating to their use is available from ATCC, SC61 and U251 brain cancer cells are available from commercial and/or sources known in the art and referenced in the scientific literature. MTT assay protocol: Day 1: cells plated in 96-well plates. Day 2: chemical agents (DMSO) are added to the wells containing cells(in triplicate). Day 3: 100 μM of MTS reagent is added to each well; ager incubating the plates for an hour, readings are taken using a spectrometer.

Small-Molecule Inhibitor Compounds—The compound C4: N′t(4-chlorophenyl)methyl]-N,N-dimethyl-N′-pyridin-2-yl-ethane-1,2-diamine (NSC 409949; Sigma C 1915, Suprastin; chloropyramine hydrochloride) is commercially available. The Y15 compound was ordered from Sigma for biochemical analyses in vitro and injection into mice for in-vivo studies. Y15 was solubilized in water at concentration of 25 mM and stored at −20° C. and -80° C.

### Example 1

Treatment of DBTRG Cells with C4

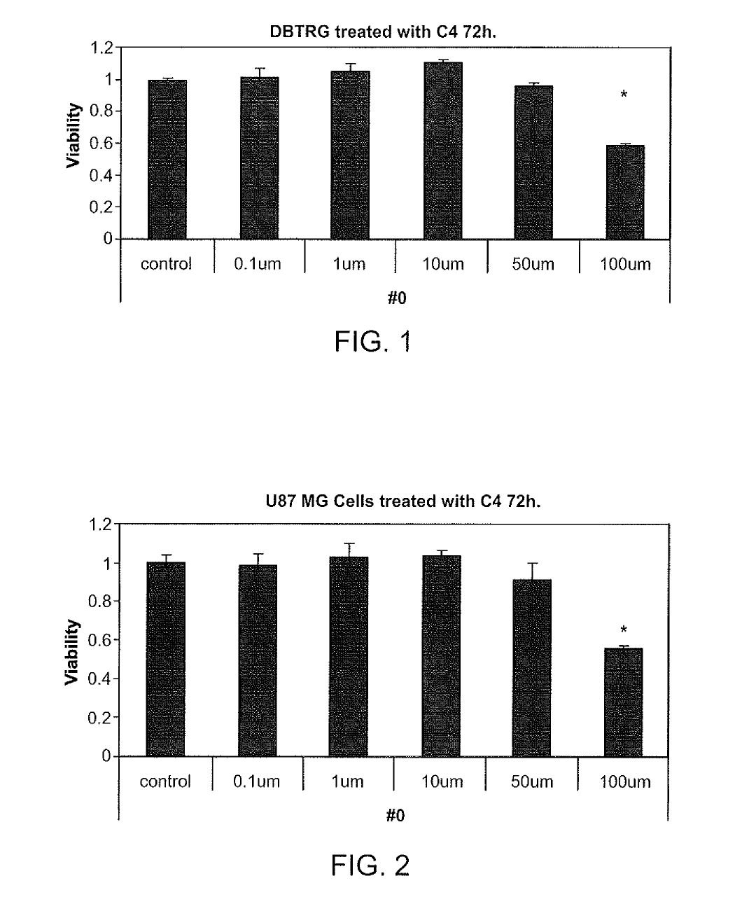

DBTRG cells were treated with C4 at various concentrations (e.g., control, 0.1 μM,1 μM, 10 μM, 50 μM, 100 μM) for 72 hours. Results are summarized in FIG. 1. C4 antiproliferative activity in DBTRG cells can be estimated based on MTS and clonogenic assays with an IC50 between 50-100 μM.

### Example 2

Treatment of U-87 Cells with C4

U-87 cells were treated with C4 at various concentrations (e.g., control, 0.1 μM, 1 μM, 10 μM, 50 μM, 100 μM) for 72 hours. Results are summarized in FIG. 2. C4 antiproliferative activity in U-87 cells can be estimated based on MTS and clonogenic assays with an IC50 between 50-100 μM.

### Example 3

Treatment of U-87 Cells with C4 and temozolomide

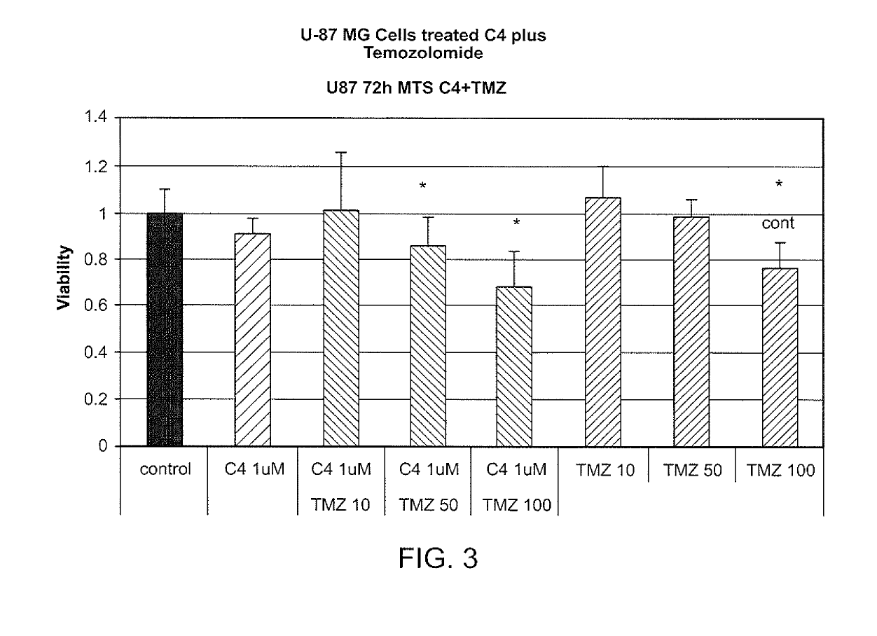

U-87 cells were treated with C4 at various concentrations (e.g., control, 1 μM), with temozolomide at various concentrations (e.g., 10 μM, 50 μM, 100 μM), and combinations of C4 at 1 with temozolomide at various concentrations (e.g., 10 μM, 50 μM, 100 μM) for 72 hours. Results are summarized in FIG. 3. Combinations of temozolomide and C4 exhibit synergistic activity in U-87 cells compared to single agent treatments.

### Example 4

Treatment of U-87 Cells with Y15 and temozolomide

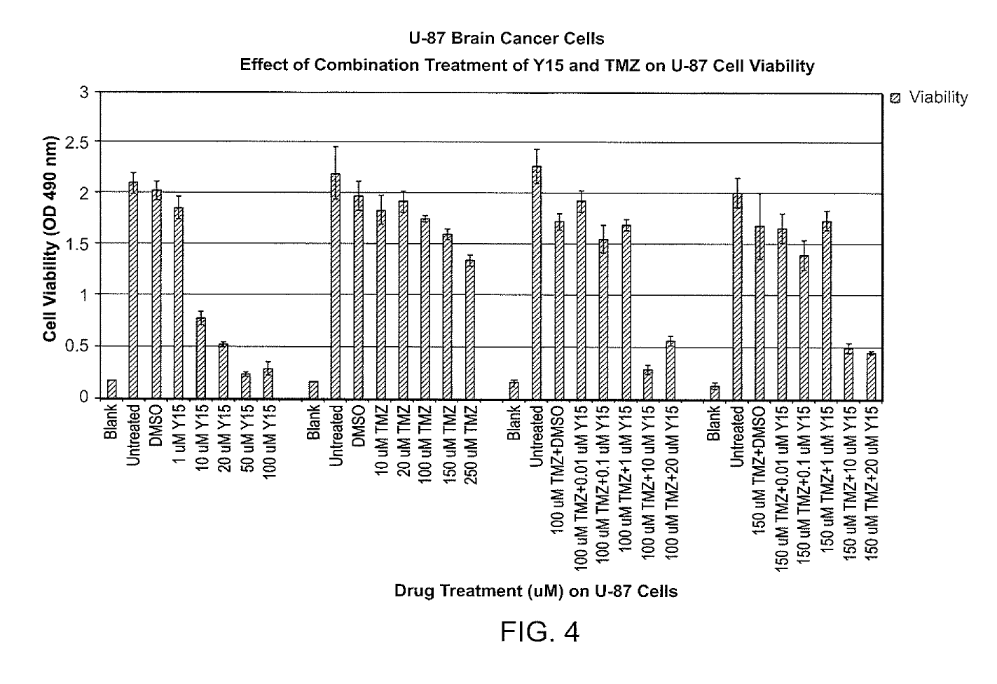

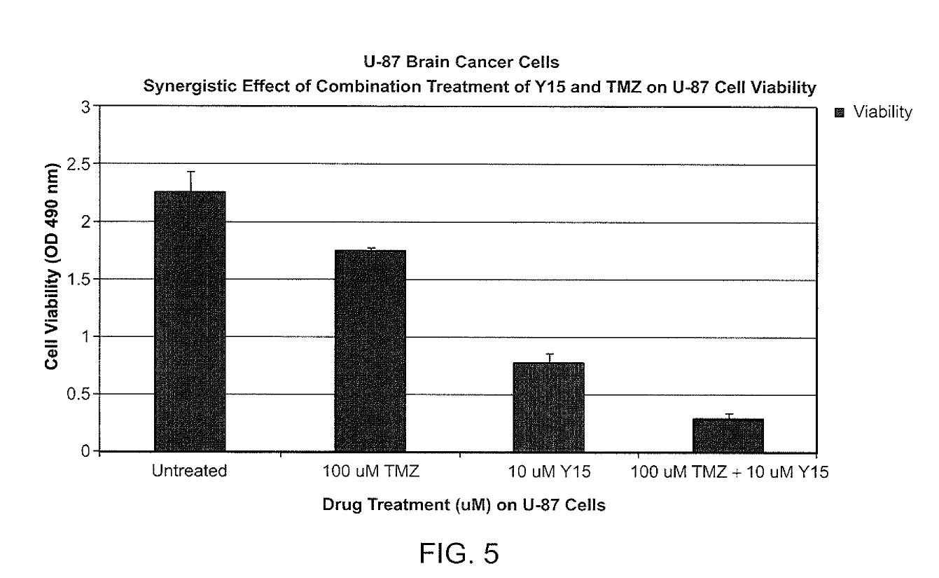

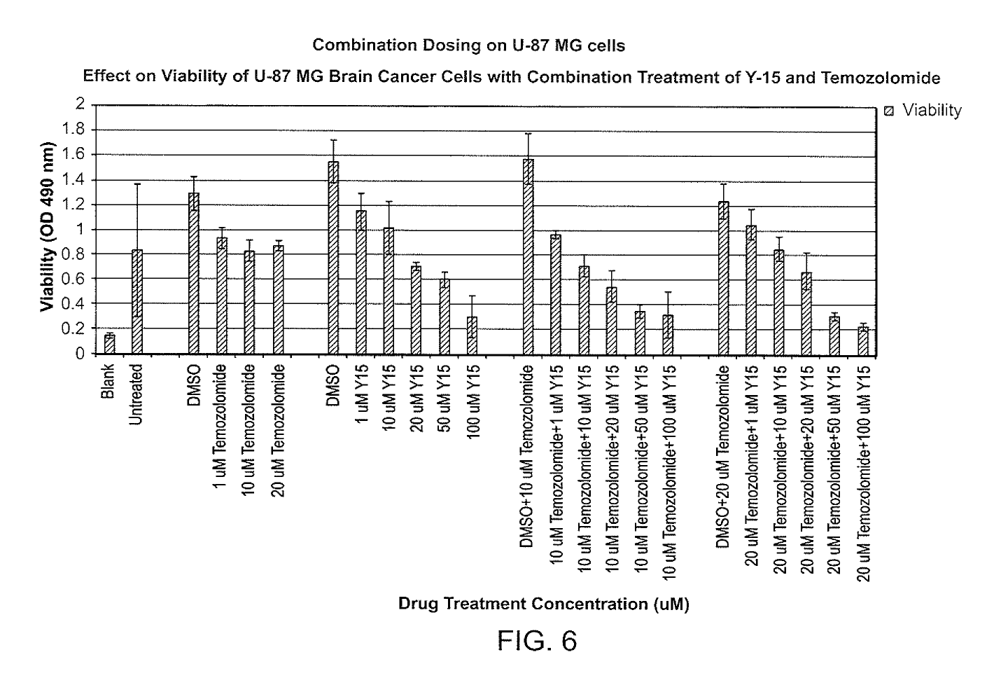

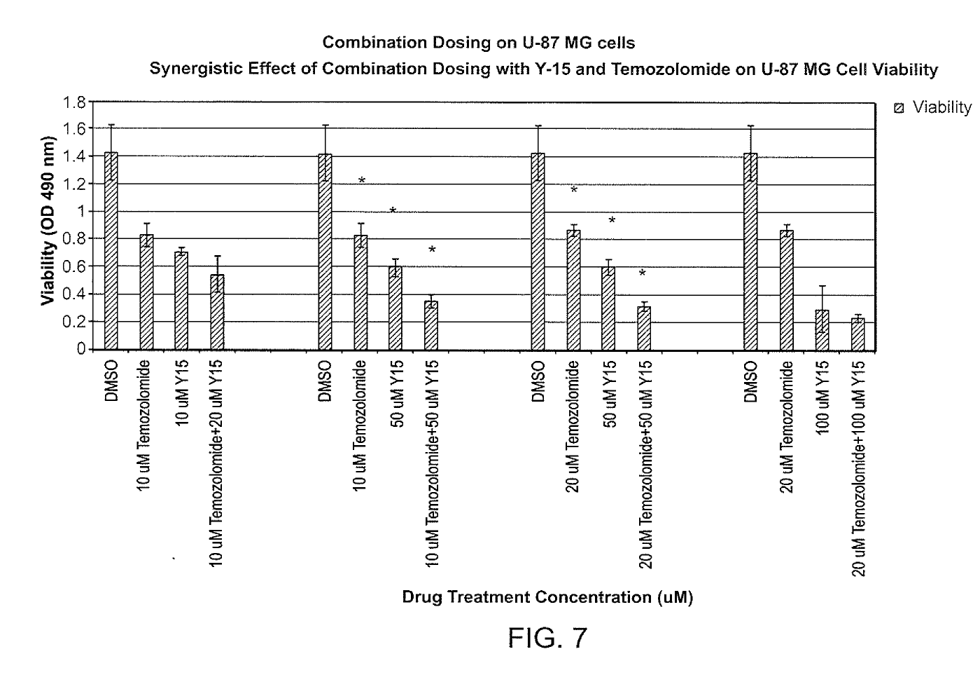

U-87 cells were treated with Y15 at various concentrations (e.g., control, 1 μM, 10 μM, 20 μM, 50 μM, 100 μM), with temozolomide at various concentrations (e.g., 10 μM, 20 μM, 100 μM, 150 μM, 250 μM), and combinations of Y15 at various concentrations (e.g., 0.01 μM, 0.1 μM, 1 μM, 10 μM, 20 μM) with temozolomide at various concentrations (e.g., 100 μM, 150 μM) for 72 hours. Results are summarized in FIGS. 4-7. Combinations of temozolomide and Y15 exhibit synergistic activity in U-87 cells compared to single agent treatments. The combinations of 10 μM Temozolomide with 50 μM Y15 and 20 μM Temozolomide with 50 μM Y15 exhibited statistical sugnificance in decreasing cell viability compared to single agent treatment at the same concentration.

### Example 5

Treatment of DBTRG Cells with Y15 and temozolomide

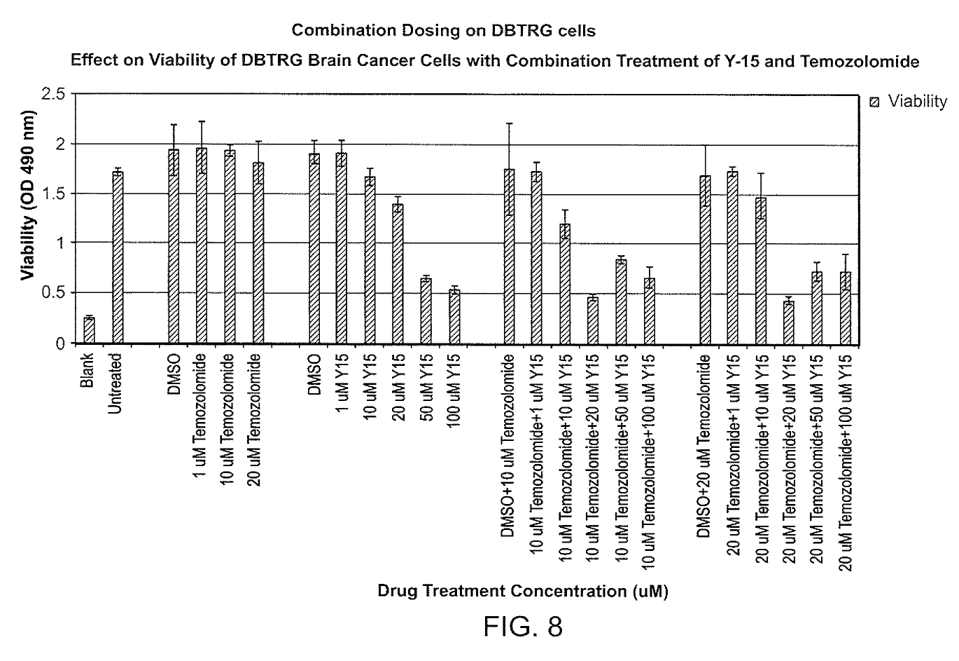

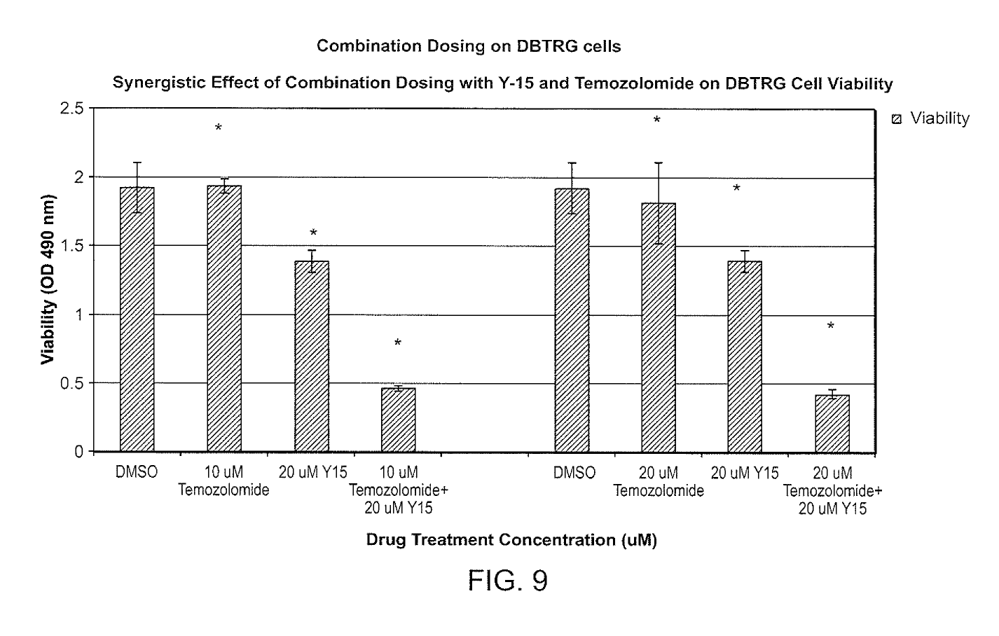

DBTRG cells were treated with Y15 at various concentrations (e.g., control, 1 μM, 10 μM, 20 μM, 50 μM, 100 μM), with temozolomide at various concentrations (e.g., 1 μM, 10 μM, 20 μM), and combinations of Y15 at various concentrations (e.g., control, 1 μM, 10 μM, 20 μM, 50 μM, 100 μM) with temozolomide at various concentrations (e.g., 10 μM, 20 μM) for 72 hours. Results are summarized in FIGS. 8-9. Combinations of temozolomide and Y15 exhibit synergistic activity in DBTRG cells compared to single agent treatments. The combinations of 10 μM Temozolomide with 20 μM Y15 and 20 μM Temozolomide with 20 μM Y15 exhibited statistical sugnificance in decreasing cell viability compared to single agent treatment at the same concentration.

### Example 6

Treatment of SC61 Cells with Y15 and temozolomide

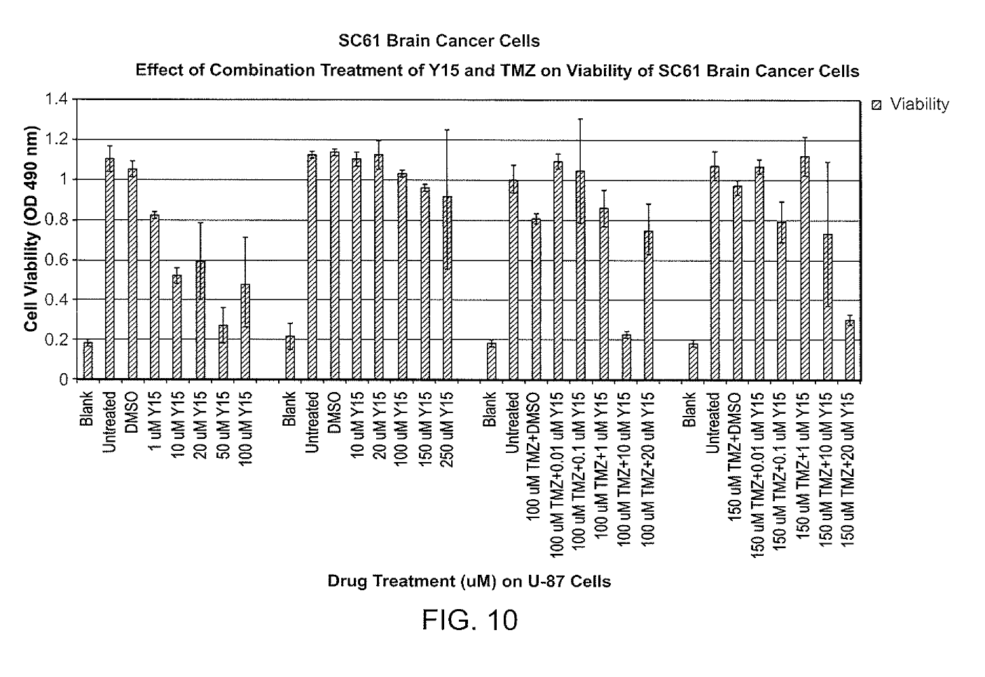

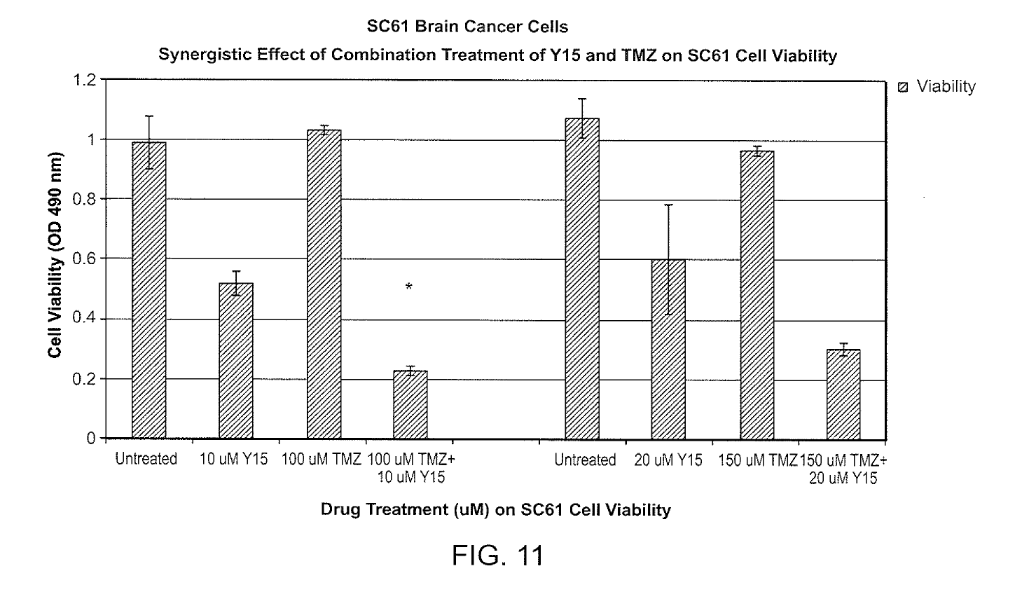

SC61 cells were treated with Y15 at various concentrations (e.g., control, 1 μM, 10 μM, 20 μM, 50 μM, 100 μM), with temozolomide at various concentrations (e.g., 10 μM, 20 μM, 100 μM, 150 μM, 250 μM), and combinations of Y15 at various concentrations (e.g., control, 0.01 μM, 0.1 μM, 1 μM, 10 μM, 20 μM) with temozolomide at various concentrations (e.g., 100 μM, 150 μM) for 72 hours. Results are summarized in FIGS. 10-11. Combinations of temozolomide and Y15 exhibit synergistic activity in SC61 cells compared to single agent treatments.

### Example 7

Treatment of U251(glioma) Cells with Y15 and temozolomide

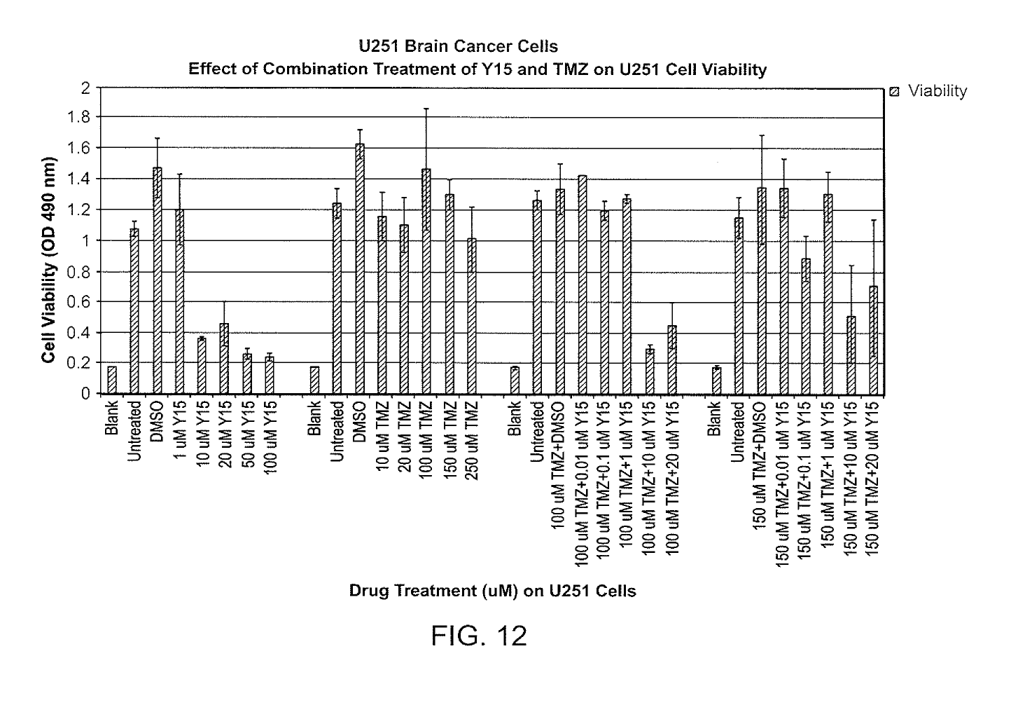

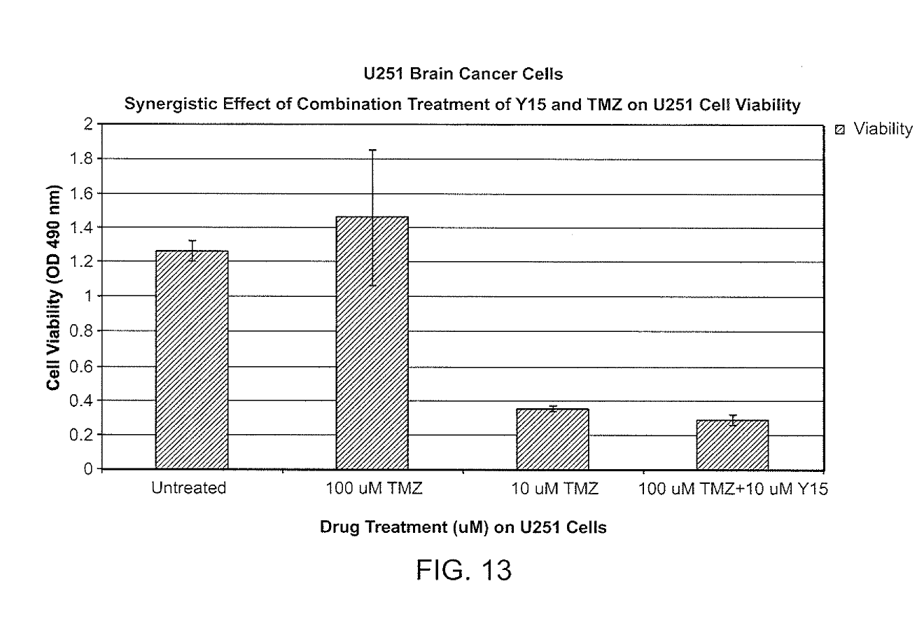

U-251 cells were treated with Y15 at various concentrations (e.g., control, 1 μM, 10 μM, 20 μM, 50 μM, 100 μM), with temozolomide at various concentrations (e.g., 10 μM, 20 μM, 100 μM, 150 μM, 250 μM), and combinations of Y15 at various concentrations (e.g., control, 0.01 μM, 0.1 μM, 1 μM, 10 μM, 20 μM) with temozolomide at various concentrations (e.g., 100 μM, 150 μM) for 72 hours. Results are summarized in FIGS. 12-13. Combinations of temozolomide and Y15 exhibit synergistic activity in U-251 cells compared to single agent treatments.

### Example 8

Toxicology Evaluation of Y15 in Mice

### Materials and Methods

### Objective of Study:

A 28-day, multiple-dose toxicologic study of the chemical 1,2,4,5-Benzenetetramine tetrahydrochloride was performed in CD-1 mice.

### Test Article:

1,2,4,5-benzenetetramine tetrahydrochloride. The test article was prepared in PBS (physiologic buffered saline). The test article was prepared by the sponsor and delivered to the study director prior to the study. The test article was stored in the freezer (0° C.), protected from light.

### Control Article:

PBS (physiologic buffered saline),
Test System:
The CD-1 [Hsd:ICR(CD-l)] albino mouse was used. The CD-1 mouse is conventionally used in 28 day toxicity tests to provide information on which human hazards can be judged, and is one of the choices of species preferred by regulatory agencies. Mice were obtained from Harlan (Frederick, MD).
Mice were acclimated for 2 days prior to testing. 12 male and 12 female mice, nulliparous and non-pregnant, were used. Normal weight gain, appearance and behavior were factors used to select healthy mice for testing. Only naive animals were used. Mice were 7 weeks old (males) and 9 weeks old (females), weighed 21-26 grams (males) and 17-23 grams (females), and were in good health at the start of the study. Mice were 11 weeks old (males) and 13 weeks old (females), weighed 20-32 grams (males) and 19-26 grams (females) at the conclusion of the study.
Mice were randomly assigned to 4 experimental groups (high-dose, medium-dose, low-dose, and controls) consisting of 3 males and 3 females. Mice were identified via cage cards, tail markings, and ear punch. Mouse identification is summarized in Table 1.
Mice were fed autoclaved Harlan-Teklad sterilizable 7012 mouse/rat diet ad libitum. The feed is routinely analyzed by the manufacturer for nutritional components and environmental contaminants. Tap water acidified to a pH of 2.5-3.5 was provided in autoclaved water bottles ad libitum. Municipal water supply is analyzed by the Bureau Veritas Consumer Product Services of Buffalo, N.Y. on an annual basis. There are no known contaminants in the feed or water provided to the test animals that would be expected to interfere with this study.
Mice were group-housed, 3 to a cage, in autoclaved polycarbonate/polysulfone microisolator cages with autoclaved contact bedding (bed ′o cobs, Anderson's Industrial Products Group).
Environmental conditions in the animal housing room were maintained at an ambient temperature of 70±3°, relative humidity of 30-70%, and a 12:12 light-dark cycle. Animal housing rooms were ventilated with 100% fresh, HEPA filtered air with 10-15 air changes per hour.

### Length of Study:

A 28-day multiple-dose toxicology study was performed.

### Test Article Preparation and Dosing:

The test article (1,2,4,5-benzenetetramine tetrahydrochloride) was prepared in PBS (the control article) and administered intraperiotoneally (IP) as 20 doses once daily on days 0-4, 7-11, 14-18, and 21-25 in the morning. The compound was diluted so as not to exceed a volume of 0.1 mL.

The test article (1,2,4,5-benzenetetramine tetrahydrochloride) was stored in the freezer (0° C.) and thawed to room temperature prior to injection. Mice were randomly allocated into 4 groups (control, low-dose, medium-dose, and high-dose) consisting of 3 males and 3 females, Dosages were calculated on a “per mouse” basis, not on a mg/kg basis. Mice in the low-dose group received the equivalent of a 15 mg/kg dose for a 20 g mouse (0.3 mg). Mice in the medium-dose group received the equivalent of a 30 mg/kg dose for a 20 g mouse (0.6 mg). Mice in the high-dose group received the equivalent of a 45 mg/kg dose for a 20 g mouse (0.9 mg). Mice in the control group received 0.1 mL of PBS (phosphate-buffered saline). Groups received the following doses, as outlined in table 1:

TABLE 1
Mouse identification codes, gender, number,
and dose of test article received.

| ID | Sex | Number | Dose |
|---|---|---|---|
| CF-1 | F | 1 | PBS |
| CF-2 | F | 2 | PBS |
| CF-3 | F | 3 | PBS |
| CM-1 | M | 1 | PBS |
| CM-2 | M | 2 | PBS |
| CM-3 | M | 3 | PBS |
| LF-1 | F | 1 | 0.3 mg |
| LF-2 | F | 2 | 0.3 mg |
| LF-3 | F | 3 | 0.3 mg |
| LM-1 | M | 1 | 0.3 mg |
| LM-2 | M | 2 | 0.3 mg |
| LM-3 | M | 3 | 0.3 mg |
| MF-1 | F | 1 | 0.6 mg |
| MF-2 | F | 2 | 0.6 mg |
| MF-3 | F | 3 | 0.6 mg |
| MM-1 | M | 1 | 0.6 mg |
| MM-2 | M | 2 | 0.6 mg |
| MM-3 | M | 3 | 0.6 mg |
| HF-1 | F | 1 | 0.9 mg |
| HF-2 | F | 2 | 0.9 mg |
| HF-3 | F | 3 | 0.9 mg |
| HM-1 | M | 1 | 0.9 mg |
| HM-2 | M | 2 | 0.9 mg |
| HM-3 | M | 3 | 0.9 mg |

### CONCLUSION

Administration of the test article was associated with pain following injection in all dose groups and with clinical signs consistent with general malaise and peritonitis in the medium- and high-dose groups of mice. Severity of clinical signs increased in a dose-dependent fashion. Administration of the test article was associated with weight loss in the medium- and high-dose groups and statistically significant lowered mean body weights in the high dose group.

Decreased body weights showed dose dependency. Injection of the test article via the intraperitoneal route resulted in gross and histopathologic peritonitis in mice in all dose groups, The severity of peritonitis and related gross lesions showed a dose-dependency with the most severe lesions being seen in the high-dose group. Some statistically and clinically significant hematologic changes were seen in mice in the high-dose group that were consistent with chronic inflammation and could be explained by the severity of peritonitis seen in this dose group. Administration of the test article was not associated with any changes in clinical chemistry that were statistically and clinically significant. Gross and histopathologic lesions were consistent with intraperitoneal administration of an irritating substance and not systemic organ pathology. Administration of the test article resulted in unexpected mortality in the high-dose group that was attributable to peritonitis. Clinical signs, body weight changes, hematology, gross and histopathologic changes, and mortality seen were all attributable to administration of an irritating substance via the IP route resulting in peritonitis.

### Example 9

Multiple-Dose Maximum Tolerated Dose (MTD) toxicologic Evaluation of Yb 15 in Mice

### Materials and Methods

### Objective of Study:

A 7-day, multiple-dose maximum tolerated dose toxicologic study of the chemical 1,2,4,5-Benzenetetramine tetrahydrochloride (Y15) was performed in CD-1 mice.

### Test Article:

The chemical 1,2,4,5-Benzenetetramine tetrahydrochloride (Y15) was the test article. Y-15 is a chemical grade compound available through Sigma-Aldrich, The test article was prepared in phosphate buffered saline (PBS). The test article was prepared by the sponsor and delivered to the study director prior to the study. The test article was stored in the freezer (0° C.), protected from light.

### Control Article:

Phosphate buffered saline (PBS).

### Test System:

The CD-1 [Hsd:ICR(CD-1)] albino mouse was used. The CD-1 mouse is conventionally used in toxicity tests to provide information on which human hazards can be judged, and is one of the choices of species preferred by regulatory agencies. Mice were obtained from Harlan (Frederick, Md.).
Mice were acclimated for a minimum of 2 days prior to testing. 20 male and 20 female mice, nulliparous and non-pregnant, were used. Normal weight gain, appearance and behavior were factors used to select healthy mice for testing. Only naive animals were used. Mice weighed 22-35 grams (males) and 20-27 grams (females), and were in good health at the start of the study. Mice were randomly assigned to 4 experimental groups (controls, low-dose, medium-dose, and high-dose) consisting of 5 males and 5 females. Mice were identified via cage cards, tail markings, and ear punch. Mouse identification codes contained information regarding experimental group (C=control, L=low-dose, M=medium-dose, H=high-dose), sex (M=male, F=female), and animal number (1-5). Individual mouse identification data is shown in appendix 1.
Mice were fed autoclaved Harlan-Teklad sterilizable 7012 mouse/rat diet ad libitum. The feed is routinely analyzed by the manufacturer for nutritional components and environmental Study contaminants. Tap water acidified to a pH of 2.5-3.5 was provided in autoclaved water bottles ad libitum. Municipal water supply is analyzed by the Bureau Veritas Consumer Product Services of Buffalo, N.Y. on an annual basis. There are no known contaminants in the feed or water provided to the test animals that would be expected to interfere with this study.
Mice were group-housed, up to 5 to a cage, in autoclaved polycarbonate/polysulfone microisolator cages with autoclaved contact bedding (bed ′o cobs, Anderson's Industrial Products Group). Environmental conditions in the animal housing room were maintained at an ambient temperature of 70±3°, relative humidity of 30-70%, and a 12:12 light-dark cycle. Animal housing rooms were ventilated with 100% fresh, HEPA filtered air with 10-15 air changes per hour.

### Length of Study:

A 7-day multiple-dose MTD toxicology study was performed.

### Test Article Preparation and Dosing:

The test article Y-15 was prepared in PBS and administered orally (PO) via orogastric gavage as a single daily dose on days 0-6 in the morning. The test article (Y-15) was diluted with the control article (PBS) so as not to exceed a volume of 100 μL. The test article was stored in the freezer (0° C.) and allowed to thaw and come to room temperature prior to administration. Mice were randomly allocated into 4 groups (controls, low-dose, medium-dose, and high-dose) consisting of 5 males and 5 females. Dosages were calculated on a “per mouse” basis, not on a mg/kg basis. Mice in the low-dose group received the equivalent of a 75 mg/kg dose for a 20 g mouse (1.5 mg). Mice in the medium-dose group received the equivalent of a 150 mg/kg dose for a 20 g mouse (3 mg). Mice in the high-dose group received the equivalent of a 300 mg/kg dose for a 20 g mouse (6 mg). Mice in the control group received 100 μL of the control article (PBS). Groups received the following doses, drug concentrations, and gavage volumes, as outlined in Table 2:

TABLE 2
Mouse groups, test article doses, test article concentrations,
test article volumes, and routes of administration for study

| Group | Dose | Concentration | Volume | Route |
|---|---|---|---|---|
| Control (PBS) | 0 mg/kg | 0 mg/mL | 100 μL | Gavage |
| Low-dose | 50 mg/kg | 10 mg/mL | 100 μL | Gavage |
| Medium-dose | 100 mg/kg | 20 mg/mL | 100 μL | Gavage |
| High-dose | 200 mg/kg | 40 mg/mL | 100 μL | Gavage |

### CONCLUSION

The MTD is defined as the highest dose that produces neither mortality nor more than a 10% decrement in body weight nor clinical signs of toxicity. Administration of the test article (Y-15) was associated with weight loss, severe clinical signs of abdominal distention, and mortality due to gastrointestinal dilatation in the high-dose group (200 mg/kg), excluding this dose as the MTD. As no mortality, clinical signs of toxicity, or decrement in mean body weight was seen in the medium-dose experimental group, it is our conclusion that the multiple-dose MTD for Y-15 administered via oral gavage in mice is at least 100 mg/kg, which was the dose tested in the medium-dose group. Additional doses of Y-15 that are greater than 100 mg/kg, but less than 200 mg/kg could be evaluated to further titrate the multiple-dose MTD if desired. As no biologically or statistically significant adverse effects were seen in the group of mice receiving the 100 mg/kg dose (medium dose) of the test article, the no observable adverse effects level (NOAEL) was determined to be 100 mg/kg. Since there were no observable adverse effects seen in the medium-dose (100 mg/kg) experimental group, it was deemed that it was not necessary to repeat testing of the low-dose (50 mg/kg) experimental group which was removed from the study due to a dosing error.

### Example 10

Single-Dose Maximum Tolerated Dose (MTD) toxicologic Evaluation of Y15 in Mice

### Materials and Methods

### Objective of Study:

A 7-day, single-dose maximum tolerated dose toxicologic study of the chemical 1,2,4,5-Benzenetetramine tetrahydrochloride (Y-15) was performed in CD-1 mice.

### Test Article:

The chemical 1,2,4,5-Benzenetetramine tetrahydrochloride (Y-15) was the test article. Y-15 is a chemical grade compound available through Sigma-Aldrich. The test article was prepared in phosphate buffered saline (PBS). The test article was prepared by the sponsor and delivered to the study director prior to the study. The test article was stored in the freezer (0° C.), protected from light.

### Control Article:

Phosphate buffered saline (PBS).

### Test System:

The CD-1 [Hsd:ICR(CD-1)] albino mouse was used. The CD-1 mouse is conventionally used in toxicity tests to provide information on which human hazards can be judged, and is one of the choices of species preferred by regulatory agencies. Mice were obtained from Harlan (Frederick, Md.).
Mice were acclimated for a minimum of 2 days prior to testing. 12 male and 12 female mice, nulliparous and non-pregnant, were used. Normal weight gain, appearance and behavior were factors used to select healthy mice for testing. Only naive animals were used. Mice were 4-7 weeks old, weighed 22-34 grams (males) and 19-24 grams (females), and were in good health at the start of the study. Mice were 5-8 weeks old, weighed 23-36 grams (males) and 20-25 grams (females) at the conclusion of the study.
Mice were randomly assigned to 8 experimental groups consisting of 3 males and 3 females which received increasing doses of the chemical Y-15. Mice were identified via cage cards, tail markings, and ear punch. Mouse identification codes contained information regarding experimental group (A=15 mg/kg dose group, B=30 mg/kg dose group, C=60 mg/kg dose group, D=90 mg/kg dose group, E=120 mg/kg dose group, F=150 mg/kg dose group, G=200 mg/kg dose group, and H=250 mg/kg dose group), sex (M=male, F=female), and animal number (1-3). Mice were fed autoclaved Harlan-Teklad sterilizable 7012 mouse/rat diet ad libitum. The feed is routinely analyzed by the manufacturer for nutritional components and environmental contaminants. Tap water acidified to a pH of 2.5-3.5 was provided in autoclaved water bottles ad libitum. Municipal water supply is analyzed by the Bureau Veritas Consumer Product Services of Buffalo, NY on an annual basis. There are no known contaminants in the feed or water provided to the test animals that would be expected to interfere with this study.
Mice were group-housed, 3 to a cage, in autoclaved polycarbonate/polysulfone microisolator cages with autoclaved contact bedding (bed ‘o cobs, Anderson's industrial Products Group).
Environmental conditions in the animal housing room were maintained at an ambient temperature of 70±3°, relative humidity of 30-70%, and a 12:12 light-dark cycle. Animal housing rooms were ventilated with 100% fresh, HEPA filtered air with 10-15 air changes per hour.

### Length of Study:

A 7-day single-dose MTD toxicology study was performed.

### Test Article Preparation and Dosing:

The test article 1,2,4,5-Benzenetetramine tetrahydrochloride (Y15) was prepared in PBS and administered orally (PO) via orogastric gavage as a single dose on day 0 in the morning. The compound was diluted so as not to exceed a volume of 0.1 mL. The test article (Y-15) was stored in the freezer (0° C.) and thawed and allowed to come to room temperature prior to administration. Mice were randomly allocated into 8 groups (A-H) consisting of 3 males and 3 females. Dosages were calculated on a “per mouse” basis, not on a mg/kg basis. Mice in group A received the equivalent of a 15 mg/kg dose for a 20 g mouse (0.3 mg). Mice in group B received the equivalent of a 30 mg/kg dose for a 20 g mouse (0.6 mg). Mice in group C received the equivalent of a 60 mg/kg dose for a 20 g mouse (1.2 mg). Mice in group D received the equivalent of a 90 mg/kg dose for a 20 g mouse (1.8 mg), Mice in group E received the equivalent of a 120 mg/kg dose for a 20 g mouse (2.4 mg). Mice in group F received the equivalent of a 150 mg/kg dose for a 20 g mouse (3.0 mg). Mice in group G received the equivalent of a 200 mg/kg dose for a 20 g mouse (4.0 mg). Mice in group 1-1 received the equivalent of a 250 mg/kg dose for a 20 g mouse (5.0 mg). Groups received the following doses, drug concentrations, and gavage volumes, as outlined in Table 3:

TABLE 3
Mouse groups, test article doses, test article concentrations,
test article volumes, and routes of administration

| Group | Dose | Concentration | Volume | Route |
|---|---|---|---|---|
| A | 15 mg/kg | 3 mg/mL | 100 μL | Gavage |
| B | 30 mg/kg | 6 mg/mL | 100 μL | Gavage |
| C | 60 mg/kg | 12 mg/mL | 100 μL | Gavage |
| D | 90 mg/kg | 18 mg/mL | 100 μL | Gavage |
| E | 120 mg/kg | 24 mg/mL | 100 μL | Gavage |
| F | 150 mg/kg | 30 mg/mL | 100 μL | Gavage |
| G | 200 mg/kg | 40 mg/mL | 100 μL | Gavage |
| H | 250 mg/kg | 50 mg/mL | 100 μL | Gavage |

### CONCLUSION

The MTD is defined as the highest dose that produces neither mortality nor more than a 10% decrement in body weight nor clinical signs of toxicity. Since mortality was seen in the highest Y-15 experimental dose group (group H −250 mg/kg), this dose should be excluded as the single dose MTD. As no mortality, adverse clinical signs, or decrement in body weight greater than 10% was seen in any of the other dose groups, it is our conclusion that the single-dose MTD for Y-15 administered via oral gavage in mice is 200 mg/kg, which was the dose tested in the second highest-dose group (group G). Based upon this single-dose oral MTD, the doses to be evaluated in a multiple-dose oral MID toxicologic study for Y-15 should include: the single dose MTD (200 mg/kg), 1/2 the single dose MTD (100 mg/kg), and 1/4 the single dose MTD (50 mg/kg).

The above results show that at 30 mg/kg-by ip administration-no toxicity, duration 28 days, maximal tolerated dose at 250 mg/kg by delivery once /week duration 7 days, and 200/mg/kg, by delivery 5 days/week duration 7 days.

### Example 11

### Tumor Growth in Xenograft Nude Nice.

Female SCID mice were purchased six weeks old and were maintained in an animal facility, and all experiments were performed in compliance with NIH animal use guidelines using IACUC protocol. The 2x10⁶ U87 cells or GL261 cells were injected into mice subcutaneously. When tumor size reached 100 mm³, drug was introduced by IP daily, 5 days per week for several weeks. Tumor diameters were measured with calipers and tumor volume in mm³ was calculated using this formula: tumor volume=(width)²×Length/2. At the end of the experiment, tumor weight and volume were determined. Tumor samples were collected for Western blotting and for immunohistochemical analysis.

Endpoints of anti-tumor efficacy include: 1) differences in tumor size between treated and control animals at termination of the experiment (T/C % and T-C4anal/T-C4%) and 2) differences in blood vessel density and lymphatic vessel density. One hour following the last dose, tumor tissues were snap frozen and Erk1/2, Akt, FAK and VEGFR-3 phosphorylation, as well as total protein levels (to ensure equal loading), were determined ex vivo by Western blot. Tumor tissues were sectioned and stained for total and phosphorylated FAK and VEGFR-3 in addition to TUNEL staining (apoptosis), Ki67 (proliferation), CD31 (angiogenesis) and LYVE1 (lymphangiogenesis). Necropsy was performed to evaluate drug toxicity.

### Data Analysis.

Experiments were repeated at least in triplicate, and data were reported as mean ±standard error of the mean. An ANOVA or student's t-test was used as appropriate to compare data between groups. Statistical significance was determined at P<0.05.

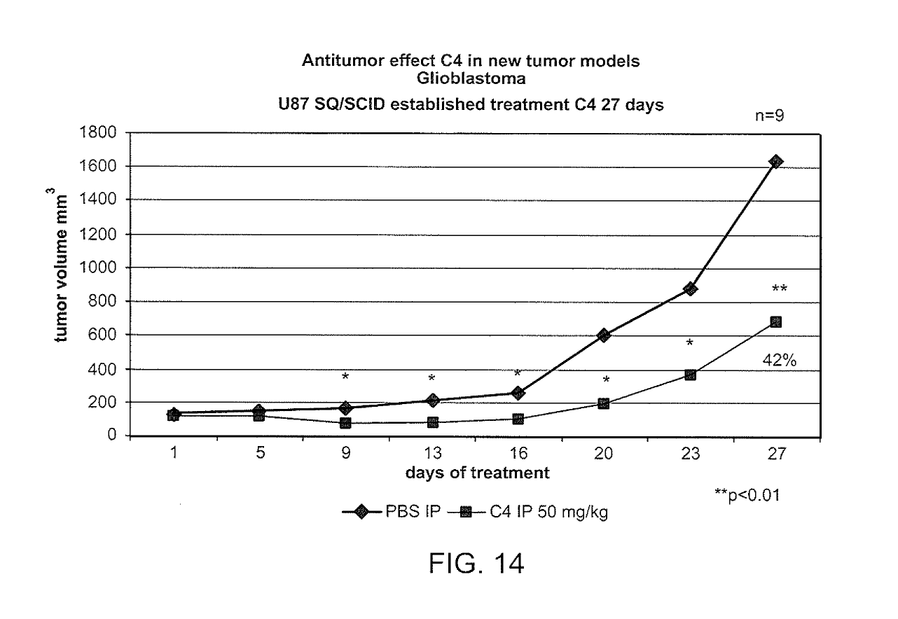

FIG. 14. C4 inhibits tumor growth in glioblastoma mouse model as a single agent—tumor volume. Treatment with intraperiotoneally (1P) administered C4 50 mg/kg started when tumor volume reached 100 mm³, after 9 days of treatment the measurable size of treated tumors was significantly smaller than in the control group. After 4 weeks of treatment, tumor volume in the treated group was 58% smaller.

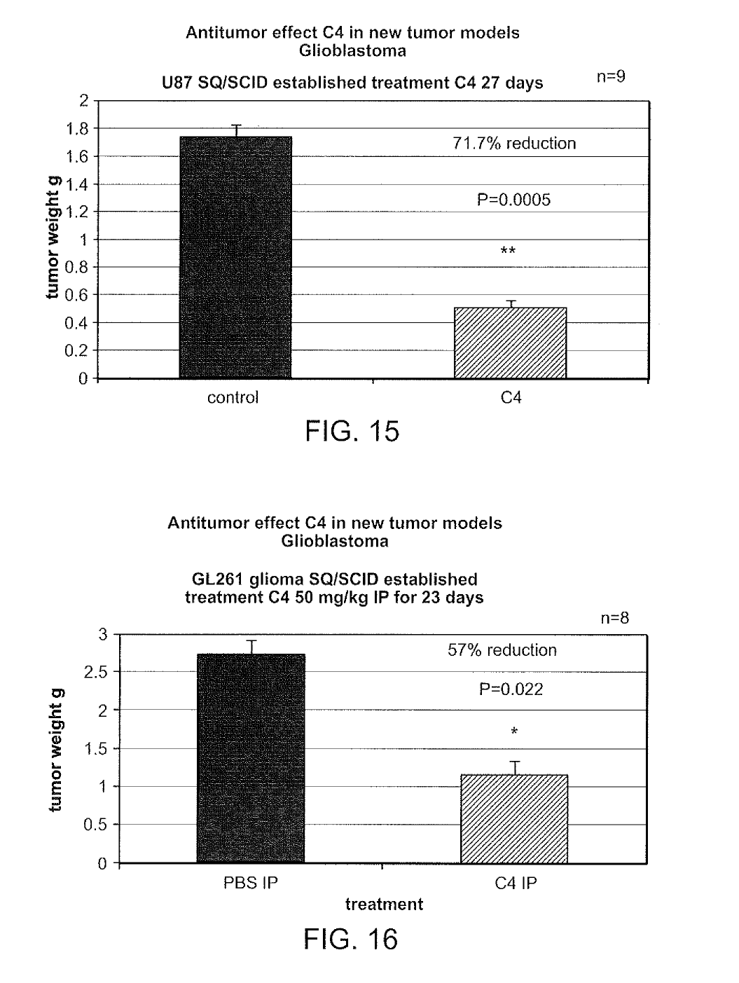

FIG. 15. C4 inhibits tumor growth in glioblastoma mouse model as a single agent—tumor weight. Treatment with IP administered C4 50 mg/kg started when tumor volume reached 100 mm³, after 27 days of treatment the mice were euthanized and excised tumors were weighed. The weight of the treated tumors was significantly smaller than in the control group. After 4 weeks of treatment tumor growth reduction reached 71.7%.

FIG. 16. C4 inhibits tumor growth in glioma singenie mouse model as a single agent—tumor weight. Treatment with 1P administered C4 50 mg/kg started when tumor volume reached 100 mm³, after 23 days of treatment the mice were euthanized and excised tumors were weighed. The weight of the treated tumors was significantly smaller than in the control group. After 3 weeks of treatment, tumor growth reduction reached 57%.

### Example 12

Multiple-Dose Maximum tolerated Dose (MTD) toxicologic Evaluation of C4 in Mice

### Materials and Methods

### Objective of Study:

A 7-day, multiple-dose maximum tolerated dose toxicologic study of the drug Suprastin (C4) was performed in CD-1 mice.

### Test Article:

The drug Suprastin (C4) was the test article. The chemical name for Suprastin is chloropyramine hydrochloride or N-[(4-chlorophenyl) methyl]-N[2-(dimethylamino) ethyl] pyridin-2-amine. The test article was prepared in sterile water. The test article was prepared by the sponsor and delivered to the study director prior to the study. The test article was stored in the refrigerator (4° C.), protected from light.
Control article:

### Sterile Water.

### Test System:

The CD-1 [Hsd:ICR(CD-1)] albino mouse was used. The CD-1 mouse is conventionally used in toxicity tests to provide information on which human hazards can be judged, and is one of the choices of species preferred by regulatory agencies. Mice were obtained from Harlan (Frederick, Md.).

Mice were acclimated for 2 days prior to testing. 20 male and 20 female mice, nulliparous and non-pregnant, were used. Normal weight gain, appearance and behavior were factors used to select healthy mice for testing. Only naive animals were used. Mice weighed 23-33 grams (males) and 19-26 grams (females), and were in good health at the start of the study. Mice weighed 22-35 grams (males) and 18-26 grams (females) at the conclusion of the study.

Mice were randomly assigned to 4 experimental groups (controls, low-dose, medium-dose, and high-dose) consisting of 5 males and 5 females. Mice were identified via cage cards, tail markings, and ear punch. Mouse identification codes contained information regarding experimental group (C=control, L=low-dose, IVI=medium-dose, H=high-dose), sex (M−male, F−female), and animal number (1-5).

Mice were fed autoclaved Harlan-Teklad sterilizable 7012 mouse/rat diet ad libitum. The feed is routinely analyzed by the manufacturer for nutritional components and environmental Study contaminants. Tap water acidified to a pH of 2.5-3.5 was provided in autoclaved water bottles ad libitum. Municipal water supply is analyzed by the Bureau Veritas Consumer Product Services of Buffalo, NY on an annual basis. There are no known contaminants in the feed or water provided to the test animals that would be expected to interfere with this study.

Mice were group-housed, up to 5 to a cage, in autoclaved polycarbonate/polysulfone microisolator cages with autoclaved contact bedding (bed ′o cobs, Anderson's Industrial Products Group). Environmental conditions in the animal housing room were maintained at an ambient temperature of 70±3°, relative humidity of 30-70%, and a 12:12 light-dark cycle. Animal housing rooms were ventilated with 100% fresh, REPA filtered air with 10-15 air changes per hour.

### Length of Study:

A 7-day multiple-dose MTD toxicology study was performed.

### Test Article Preparation and Dosing:

The test article Suprastin (C4) was prepared in sterile water and administered orally (PO) via orogastric gavage as a single daily dose on days 0-6 in the morning. The compound was diluted with the control article (sterile water) so as not to exceed a volume of 100 μM. The test article (Suprastin) was stored in the refrigerator (4° C.) and allowed to come to room temperature prior to administration. Mice were randomly allocated into 4 groups (controls, low-dose, medium-dose, and high-dose) consisting of 5 males and 5 females. Dosages were calculated on a “per mouse” basis, not on a mg/kg basis. Mice in the low-dose group received the equivalent of a 75 mg/kg dose for a 20 g mouse (1.5 mg). Mice in the medium-dose group received the equivalent of a 150 mg/kg dose for a 20 g mouse (3 mg). Mice in the high-dose group received the equivalent of a 300 mg/kg dose for a 20 g mouse (6 mg). Mice in the control group received 100pL of the control article (sterile water). Groups received the following doses, drug concentrations, and gavage volumes, as outlined in Table 4:

TABLE 4
Mouse groups, test article doses, test article concentrations,
test article volumes, and routes of administration

| Group | Dose | Concentration | Volume | Route |
|---|---|---|---|---|
| Control | Sterile water | N/A | 100 μL | Gavage |
| Low-dose | 75 mg/kg | 15 mg/mL | 100 μL | Gavage |
| Medium-dose | 150 mg/kg | 30 mg/mL | 100 μL | Gavage |
| High-dose | 300 mg/kg | 60 mg/mL | 100 μL | Gavage |

### CONCLUSION

The MTD is defined as the highest dose that produces neither mortality nor more than a 10% decrement in body weight nor clinical signs of toxicity. Administration of the test article in the high-dose group was associated with weight loss, severe clinical signs of abdominal distention, and mortality due to gastrointestinal dilatation in the high-dose group (300 mg/kg), excluding this dose as the MTD. As no mortality or decrement in mean body weight was seen in the low- or medium-dose groups, and the only clinical signs seen (slight ruffled fur) were extremely mild and brief in duration, it is our conclusion that the multiple-dose MTD for Suprastin (C4) administered via oral gavage in mice is at least 150 mg/kg, which was the dose tested in the medium-dose group.

### REFERENCES

1. Schaller, M. D. (1996) J Endocrino1150(1), 1-7

2. Hildebrand, J. D., Schaller, M. D., and Parsons, J. T. (1993) Journal of Cell Biology 123(4), 993-1005
3. Xing, Z., Chen, H. C., Nowlen, J. K., Taylor, S. J., Shalloway, D., and Guan, J. L. (1994) Mol Biol Cell 5(4), 413-421
4. Cobb, B. S., Schaller, M. D., Leu, T. H., and Parsons, J. T. (1994) Molecular & Cellular Biology 14(1), 147-155
5. Schaller, M. D., Hildebrand, J. D., Shannon, J. D., Fox, J. W., Vines, R. R., and Parsons, J. T. (1994) Molecular & Cellular Biology 14(3), 1680-1688
6. Hanks, S. K., and Polte, T. R. (1997) Bioessays 19(2), 137-145
7. Golubovskaya, V. M., and Cance, W. G. (2007) Int Rev Cytol 263, 103-153
8. Smith, C. S., Golubovskaya, V. M., Peck, E., Xu, L. H., Monia, B. P., Yang, X., and Cance, W. G. (2005) Melanoma Res 15(5), 357-362
9. Xu, L.-h., Yang, X.-h., Bradharn, C. A., Brenner, D. A., Baldwin, A. S., Craven, R. J., and Cance, W. G. (2000) J. Biol. Chem. 275, 30597-30604
10. Golubovskaya, V., Beviglia, L., Xu, L. H., Earp, H. S., 3rd, Craven, R., and Cance, W. (2002). J Biol Chem 277(41), 38978-38987
11. Haider, J., Landen, C, N., Jr., Lutgendorf, S. K., Li, Y., Jennings, N. B., Fan, D., Nelkin, G. M., Schmandt, R., Schaller, M. D., and Sood, A. K. (2005) Clin Cancer Res 11(24 Pt 1), 8829-8836
12. Beierle, E. A., Trujillo, A., Nagaram, A., Kurenova, E. V., Finch, R., Ma, X., Vella, J., Cance, W. G., and Golubovskaya, V. M. (2007) J Biol Chem 282(17), 12503-12516
13. McLean, G. W., Carragher, N. O., Avizienyte, E., Evans, J., Brunton, V. G., and Frame, M. C. (2005) Nat Rev Cancer 5(7), 505-515
14. van Nimwegen, M. J., and van de Water, B. (2006) Biochem Pharmacol
15. Golubovskaya, V. M., Virnig, C., and Cance, W. G. (2007) Mol Carcinog
16. Liu, T. J., LaFortune, T., Honda, T., Ohmori, 0., Hatakeyama, S., Meyer, T., Jackson, D., de Groot, J., and Yung, W. K. (2007) Mol Cancer Ther 6(4), 1357-1367
17. Slack-Davis, J. K., Martin, K. H., Tilghman, R. W., Iwanicki, M., Ung, E. J., Autry, C., Luzzio, M. J., Cooper, B., Kath, J. C., Roberts, W. G., and Parsons, J. T. (2007)J Biol Chem 282(20), 14845-14852
18. Haider, J., Lin, Y. G., Merritt, W. M., Spannuth, W. A., Nick, A. M., Honda, T., Kamat, A. A., Han, L. Y., Kim, T. J., Lu, C., Tari, A. M., Bornmann, W., Fernandez, A., Lopez-Berestein, G., and Sood, A. K. (2007) Cancer Res 67(22), 10976-10983
19. Sandberg, E. M., Ma, X., He, K., Frank, S. J., Ostrov, D. A., and Sayeski, P. P. (2005) J Med Chem 48(7), 2526-2533
20. Ceccarelli, D. F., Song, H. K., Poy, F., Schaller, M. D., and Eck, M. J. (2006) J Biol Chem 281(1), 252-259
21. Golubovskaya, V. M., Finch, R., Kweh, F., Massoll, N. A., Campbell-Thompson, M., Wallace, M. R., and Cance, W. G. (2007) Mol Carcinog , Septe,ber 11[Epub]
22. Golubovskaya, V. M., Finch, R., and Cance, W. G. (2005) J Biol Chem 280(26), 25008-25021

The disclosures of each and every patent, patent application and publication cited herein are hereby incorporated herein by reference in their entirety.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

1. A method of treating a subject suffering from or susceptible to a brain cancer comprising administering to the subject in need thereof a therapeutically effective amount of the compound C4 or Y15.

2. (canceled)

3. A method of treating a subject suffering from or susceptible to melanoma comprising administering to the subject in need thereof a therapeutically effective amount of the compound C4 or Y15.

4. (canceled)

5. The method of claim 1 further comprising administering to the subject in need thereof a therapeutically effective amount of temozolomide.

6. The method of claim 5 wherein the compounds are administered separately.

7. The method of claim 5 wherein the compounds are administered concurrently.

8. The method of claim 5 wherein the compounds are administered in a single-dose form.

9. A composition comprising temozolomide and a second compound Wherein the second compound is:
C4: N′-[(4-chlorophenyl)methyl]-N,N-dimethyl-N′-pyridin-2-yl-ethane-1,2-diamine (NSC 409949; Sigma C1915, Suprastin; chloropyramine hydrochloride); or
Y15: 1,2,4,5-benzenetetraamine tetrahydrochloride.

10. The composition further comprising a pharmaceutically acceptable carrier.

11. The composition of claim 9 in a single-dose formulation.

12. (canceled)

13. (canceled)
